(19) United States
(12) Patent Application Publication
(10) Pub. No.: US 2021/0010942 A1
(43) Pub. Date: Jan. 14, 2021

(54) COLORIMETRIC SENSOR FOR DETECTION OF FOOD SPOILAGE

(71) Applicant: WASHINGTON STATE UNIVERSITY, Pullman, WA (US)

(72) Inventors: Shyam SABLANI, Pullman, WA (US); Barbara RASCO, Pullman, WA (US)

(21) Appl. No.: 16/918,327

(22) Filed: Jul. 1, 2020

Related U.S. Application Data
(60) Provisional application No. 62/869,467, filed on Jul. 1, 2019.

(51) Int. Cl. G01N 21/78; G01N 33/00; G01N 33/14; G01N 33/04

(52) U.S. Cl. CPC G01N 21/783; G01N 33/04; G01N 33/143; G01N 33/0047

(57) ABSTRACT

Colorimetric sensors attached to food packaging and exposed to the packaging headspace allow supply chain managers and consumers to monitor food freshness and expected shelf life based on food condition without unsealing containers or relying on the human senses of smell or taste. A gas-permeable membrane prevents direct food contact with sensor material, and color changes indicate increased concentration of volatile organic compounds generated during spoilage of the food spoilage. Color change may be read by machine or eye. One application uses silicon dioxide nanoparticles coated with Schiff's reagent and embedded in milk carton screw caps to detect spoilage to address variations in shelf life due to temperature abuse within the distribution system.

### CROSS-REFERENCE TO RELATED APPLICATIONS

The present application claims, under 35 U.S.C. § 119, the priority benefit of U.S. Provisional Application No. 62/869,467, filed Jul. 1, 2019, entitled “COLORIMETRIC SENSOR FOR DETECTION OF FOOD SPOILAGE,” which is incorporated herein by reference in its entirety.

### FEDERALLY SPONSORED RESEARCH

This invention was made with government support under grant no. 2018-31100-06053 awarded by the United States Department of Agriculture—National Institute of Food and Agriculture. The government has certain rights in the invention.

### TECHNICAL FIELD

This relates to food packaging, date marking, storage, and distribution, specifically spoilage indicators on perishable products to reduce food waste and ensure consumer safety.

### BACKGROUND

The USDA Economic Research Service has estimated a yearly loss of more than ninety-six billion pounds of food in the U.S. by retailers, foodservice, and consumers, with fluid milk accounting for nearly 20% of this loss. Date marking highly perishable packaged products provides some consumer protection, but approximation inherent in this approach results in both purchase of some spoiled food and destruction of some safe foods. Shelf-life inconsistencies are amplified in warmer regions and seasons, as refrigeration systems are strained, and even short temperature excursions on exposed loading docks accelerate spoilage. Food products maintained in optimal conditions may remain safe for consumption beyond dates marked at the time of packaging, resulting in disposal of food which would be safe for consumption.

Microbial growth with respect to food spoilage can be determined by a number of traditional methods based on single viable cells. Although these traditional methods of detection are sensitive and inexpensive, they require several days to generate results. Thus, rapid detection of bacterial growth using an effective but prompt indicator could effectively control food spoilage, in particular, milk quality and ensure product safety. Recently, several powerful spoilage detection tools have been developed. These commercial tools include polymerase chain reaction, as well as molecular, biological, and immunological and/or DNA-based detection. However, these methods are limited by long culture times, the need for highly trained laboratory personnel, and the need for expensive equipment with high maintenance requirements.

Accordingly, a need exists for spoilage indicators in packaged foods to visually determine the amount of shelf life remaining and whether a product has spoiled. Such a sensor must be accurate, cost effective, easy in operation, rapid, and reliable in determining and displaying shelf life. The embodiments herein are directed to such a need.

### SUMMARY OF THE INVENTION

A first aspect of the invention is directed to a sensing device for detecting food spoilage in a package, that includes: a substrate; one or more reagents coupled to the substrate surface; a vessel configured with a viewing window and a gas-permeable membrane, wherein the vessel is arranged to position the substrate adjacent to a food content within the package, and wherein the gas-permeable membrane is therebetween the substrate and the food content within the package; and wherein the one or more reagents provides a visual indication of spoilage of the food content.

Another aspect of the invention is directed to a disposable sensing device for detecting food spoilage that includes: a substrate; one or more reagents coupled to the substrate surface; a vessel configured with a viewing window and a gas-permeable membrane, wherein the vessel is arranged to position the substrate adjacent to a food content within a container, and wherein the gas-permeable membrane is therebetween the substrate and the food content within the container; and a means of removably coupling the vessel to the container.

For pasteurized milk and other foods, bacterial spoilage depends upon raw product quality, processing methods, and conditions during storage, retail marketing, transportation, and handling. Volatile organic compounds (VOCs) produced by spoilage microflora are responsible for undesirable odors and flavors. Compounds including aldehydes, ketones, and alcohols are metabolic byproducts of milk spoilage, and can also be formed through chemical oxidation.

Colorimetric sensors disclosed herein are coupled (i.e., mounted) to food packages, with a semi-permeable membrane (a gas-permeable membrane) separating the sensor core—a substrate coated with reagent—from the headspace or food content within an individual package. Such a membrane structure disclosed herein often is configured of pore diameters to permit diffusion of desired target molecules while simultaneously excluding larger non-target molecules. Sensors change color when exposed to sufficient concentrations of specific spoilage-related VOCs. Sensors can then be read by consumers and handlers within the food distribution system, using eye or vision systems, even as packages remain sealed. This in turn facilitates early detection of spoilage due to product variability and inconsistent temperature control during transport and storage.

The preferred embodiment herein is a package-based sensor that detects degrees of food spoilage, which includes a core solid substrate material providing a base for one or more colorimetric reagents. This core sensor material is held adjacent to the headspace or food content of the package. A gas-permeable membrane prevents liquid and solid contact between the sensor material and food product, while allowing target VOCs to reach the sensor material.

A clear non-gas-permeable membrane provides a window for viewing the core sensor material. Substrates can include nanoparticles, papers, fibers, polymers, and polymer sheets. The gas-permeable membrane can include polyethylene and polypropylene. Silicon dioxide (SiO₂) nanoparticles can be selected as the substrate to provide greater exposed surface area than a smooth surfaced flat sheet substrate. For milk spoilage, Schiff's reagent can be affixed to the substrate.

The core sensor material can be contained in a pocket between the gas-permeable membrane and window material. Alternately, it can be held in a more rigid material, such as plastic forming a threaded screw cap. The sensor device can be alternately mounted adjacent to the headspace of a package, but separate from the package closure system. In any case the gas-permeable membrane is exposed to the headspace, and can be mounted to traditional plastic milk bottles and coated paper cartons.

In other embodiments, sensors can be attached to bulk containers or used by home consumers once factory-sealed packages are opened. This can include replacements for factory installed caps. Similarly, sensors can be attached to modified human breast-milk containers to help caregivers manage supplies used to feed infants. For these applications, a disposable sensing device includes a core solid substrate material providing a base for one or more colorimetric reagents. This core sensor material is held in a vessel and has a means such as a peel and stick adhesive to attach it to a container such that a gas-permeable membrane separates the sensor core material from the contents of the container, preventing liquid and solid contact while allowing target VOCs to reach the sensor material.

### BRIEF DESCRIPTION OF FIGURES

FIG. 1A shows a colorimetric screw-cap example sensor location for typical milk and juice containers.

FIG. 1B shows another colorimetric screw-cap example sensor location for typical milk and juice containers.

FIG. 1C illustrates a surface mount coupled to a carton closure.

FIG. 2 shows testing apparatus wherein Schiff's reagent coated nanoparticles are exposed to milk VOCs through filter paper.

FIG. 3A shows magnified images of uncoated SiO₂ nanoparticles.

FIG. 3B shows Schiff's reagent coated SiO₂ nanoparticles.

FIG. 3C shows SiO₂ nanoparticles reacted with VOCs in spoiled milk.

FIG. 4 shows aerobic counts over time in milk.

FIG. 5 depicts color changes of Schiff's reagent coated nanoparticles over time for milk at four storage temperatures, wherein the ΔE scale is used to quantify differences between colors

FIG. 6 shows aerobic counts and color change of sensor over milk shelf life at 19° C.

FIG. 7 shows aerobic counts and color change of sensor over milk shelf life at 15° C.

FIG. 8 shows aerobic counts and color change of sensor over milk shelf life at 13° C.

FIG. 9 shows aerobic counts and color change of sensor over milk shelf life at 7° C.

FIG. 10 shows correlation between aerobic counts and color change of sensor over time at four temperature profiles.

FIG. 11 shows a cross-section of one example embodiment where sensor is integral with a screw cap of a food package.

FIG. 12A shows a cross-section of an example rigid plastic form sensor mount which can be bonded to package separate from its closure system.

FIG. 12B shows a cross-section of an example flexible mount which can be bonded to package separate from its closure system.

FIG. 13A shows an example embodiment wherein the sensor is mounted on a sealed food carton which does not have a cap closure.

FIG. 13B shows an example embodiment wherein the sensor is mounted on a carton which has a separate cap.

FIG. 13C shows an example embodiment wherein the sensor is incorporated into the cap.

FIG. 14 shows an example package label where the sensor color may be compared to a spectrum of printed colors.

### DETAILED DESCRIPTION OF THE INVENTION:

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Multiple methods are available to bond and hermetically seal materials, including but not limited to adhesives and plastic welding. Figures and description herein will generally not specify types of bonds, but practitioners skilled in the art will recognize that adjacent layers of material are understood to be hermetically sealed together.

### Specific Description

Embodiments of the sensors herein generally include a colorimetric material (e.g., a reagent) separated from the contents of a food package by a gas-permeable membrane which prevents direct liquid or solid food contact. Moreover, it is also to be noted that a reagent for the present invention includes any substance or compound which, when exposed to a target VOC, chemically reacts with the VOC to produce a detectable colorimetric change. It is also to be appreciated that with respect to milk or juice cartons, jugs, etc., the separation often includes a headspace that contains VOC diffusion effluents that is monitored by the sensors disclosed herein. While such a headspace is a beneficial aspect of the embodiments of the present invention, resultant VOC's can also be detected colorimetrically for those packaged products without a headspace, based on detectable effluents such sensors are configured to interrogate.

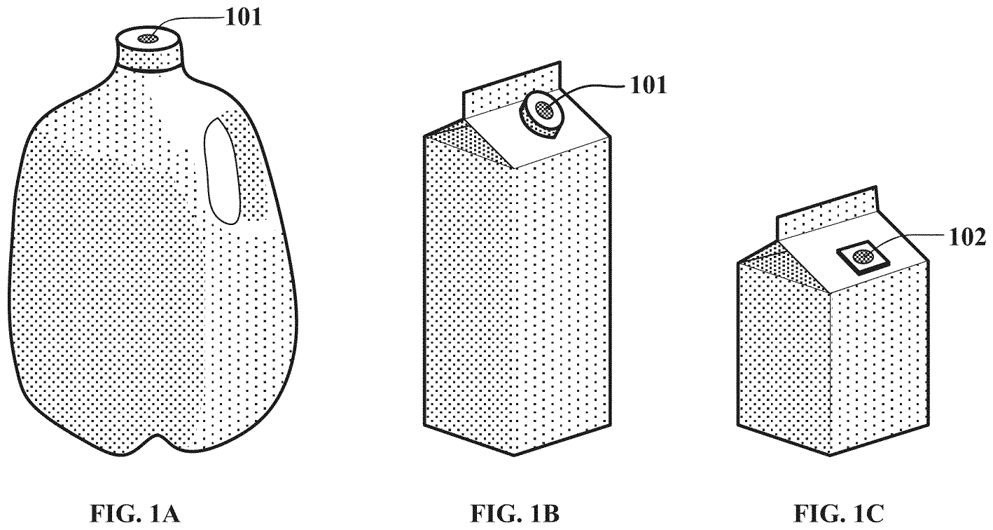

Turning to the figures, FIG. 1A shows a cap mounted sensor 101 on a food package container, such as, for example a plastic milk container or a juice container (jug). A similar sensor can be used on cartons with caps, as in FIG. 1B. For cartons without caps, such as those which are opened by tearing seams of the folded top, FIG. 1C shows a surface mount sensor 102. A surface mount can also be used along with caps, such as where a non-permeable seal is employed under the cap which would prevent sensor function.

It is to be noted that while functionalized nanoparticles with a Schiff's reagent is a preferred and beneficial aspect of the embodiments herein, it is also to be appreciated that other sensors having a particular reagent can also be contained within packaged food products, including those which potentially provide higher fidelity than batch date-stamping.

### Milk Sensor Development and Fabrication

A milk freshness sensor using an SiO₂ nanoparticle substrate coated with Schiff's reagent has been developed and tested to detect milk spoilage within various storage temperature profiles. The specific formulation is not intended to limit this disclosure's applicability with the use of other colorimetric reagents, such as, for example hypochlorites. This work has validated that the colorimetric nanosensor core can be used to monitor the formation of volatiles during spoilage of food items by detection of VOCs in the package headspace. Bacterial growth produces aldehydes, volatile acids, alcohols, ketones, and other chemical substances, and the sensor color changes from pink to purple in the presence of increasing VOC concentrations. As an example, to detect aldehydes, the indicator material (e.g., Schiff's base reagent coupled to the substrate, e.g., SiO₂ nanoparticles) operating within a pH range (e.g., 5-6.8), enables a visual detection of a given concentration of the chemical substance by way of a resultant Magenta color. To detect, for example, guaiacol and other phenols, the colorimetric reagents coupled to a substrate (e.g., SiO₂ nanoparticles) for detection can include hypochlorites.

SiO₂ is a known food and food packaging additive and silica-based nanoparticles have a large surface area: typical particles sized 10-40 nm have a specific surface area in the 25-105 m²/g range. Nanosized SiO₂ particles are available in ultra-high purity, coated, hydrophilic, lipophilic, and dispersed forms. Nanosilica has a reactive surface that readily interacts with chemical compounds, making it work as a matrix for which a wide range of compounds could be associated.

Using SiO₂ nanopowder/nanoparticles (99+ size 20-30 nm, amorphous) (US Research Nanomaterials, Inc.) and Schiff's reagent (Sigma-Aldrich), a total of 3 mL of Schiff's reagent was mixed with 1 g of silicon dioxide nanoparticles and placed into a non-reactive dish and held for 48 hours under a fume hood to evaporate the moisture. Coated pink nanoparticles were then collected and ground to separate agglomerated particles—the core sensor material—for further use.

### Response at Different Temperature Profiles

Descriptions herein of methods to measure VOCs and bacterial growth explain validation of one embodiment and are not intended to limit claim scopes. Other testing and analytical methods can be used in designing other embodiments.

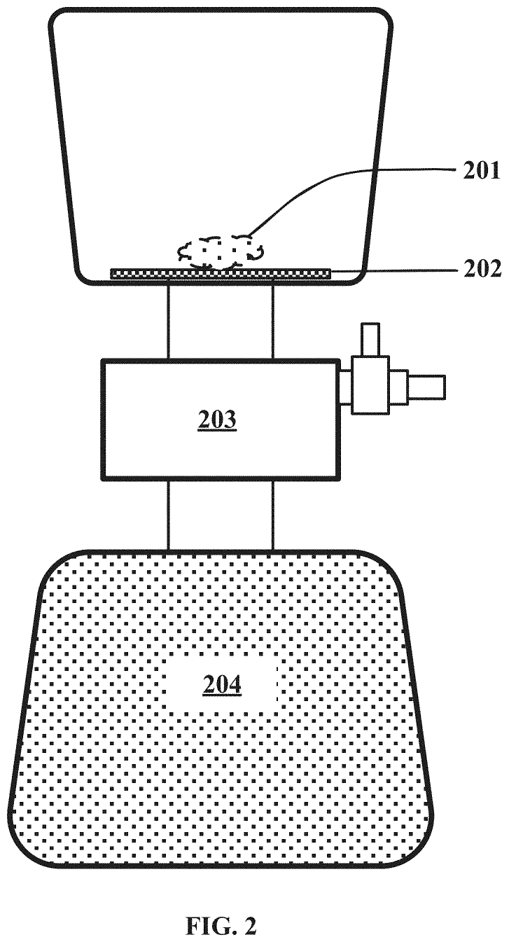

To test the sensor core material, fresh pasteurized whole cow's milk was placed in 250 mL sterile glass bottles with filters. Milk stored at 13, 15, and 19° C. was analyzed every 12 hours, while milk stored at 7° C. were measured every 3 days. Color change in response to exposure to VOCs was measured. The SiO₂ nanoparticles coated with Schiff's reagent (>0.75 gm) were placed on a layer of filter paper (55 mm, 2×2.5 cm) on top of a 250 ml filtration device. During the experiments, milk was placed in a sterile glass bottle underneath the filtration device. As illustrated in FIG. 2, the sensor nanoparticles 201 rest on a filter paper 202 exposed to VOCs from a milk sample 204 via the filtration device 203.

Color change in nanoparticles was measured as reflectance using a tristimulus color meter (Minolta Chroma CR-200, Minolta Co.), an instrument that mimics how humans perceive color and color changes. Color was measured before the reaction began (zero time). Colorimetric response was quantified by measuring the color from pink to purple. The colorimeter was standardized against a white tile before each measurement set. Measurements were taken from within the funnel to avoid stray light. The L* (lightness), a* (red to green), and b* (blue to yellow) values were measured to describe the color of the indicator. The total color change (ΔE) was calculated with the formula ΔE=\sqrt{(L*−L₀*)²+(a*−a₀*)²+(b*−b₀*)²}.

ΔE is generally used to distinguish between two colors, as indicated by the following scale: ΔE<3 (no perceptible difference); 3<ΔE<6 (very small difference); 6<ΔE<9 (fairly perceptible difference); 9<ΔE<12 (perceptible difference); and ΔE>12 (different colors).

### Determination of Bacterial Counts

Microbial analysis was carried out according to Standard Methods for the Examination of Dairy Products (SMEDP). Decimal dilutions of the pasteurized milk were prepared in peptone water (0.1% peptone; Oxoid, Basingstoke, UK), with duplicate 1 mL samples of appropriate dilutions spread on non-selective agar plates. Dilutions were plated and incubated as follows: aerobic bacterial count (ABC) on plate count agar (PCA) was incubated aerobically at 32° C. for 3 days; psychrotrophic bacteria on PCA was incubated aerobically at 7° C. for 10 days; and dilutions on plate count agar were incubated aerobically at 32° C. for 24 h. All samples were plated in duplicate. Bacterial populations (log₁₀ CFU/mL) shown were the average of two replicates.

### VOC Analysis

VOC concentrations in milk were determined using gas chromatography (GC) and solid phase microextraction (SPME). Milk (2 mL) was placed in a 4.0 mL vial containing 0.65 g of NaCl (to enhance VOC activity and concentration in the headspace), and was then stirred on a stirring plate. An SPME device with fiber-coated (75 um) carboxen polydimethylsiloxane (CAR/PDMS) fused silica was exposed to the headspace for approximately 60 min before GC injection. Spitless injection was carried out for 3 min at 200° C. into a Hewlett-Packard 589011/5970 GC/MSD equipped with a DB-1 60-m column. Transfer line temperatures and ion sources were held at 250° C. Helium was used as the carrier gas, and the injector and detector temperatures were both 200° C. The column temperature was initially held at 30° C. for 5 min and then increased to 50° C. at a rate of 2° C./min. The sample was desorbed for 5 min using an SPME liner.

### Statistical Analysis

The mean and standard deviation are used to summarize the log₁₀ viable aerobic plate counts, VOC concentrations, and ΔE values. A linear relationship between ΔE values and log₁₀ viable aerobic plate counts were determined and R² calculated. Statistical differences in regression slopes were calculated using an analysis of covariance. P-values were adjusted for multiple comparisons using Hochberg's method. Statistical analyses were conducted in JMP v. 13.0 (SAS) and SAS v. 9.4 (SAS).

### Test Results—Microbial Growth

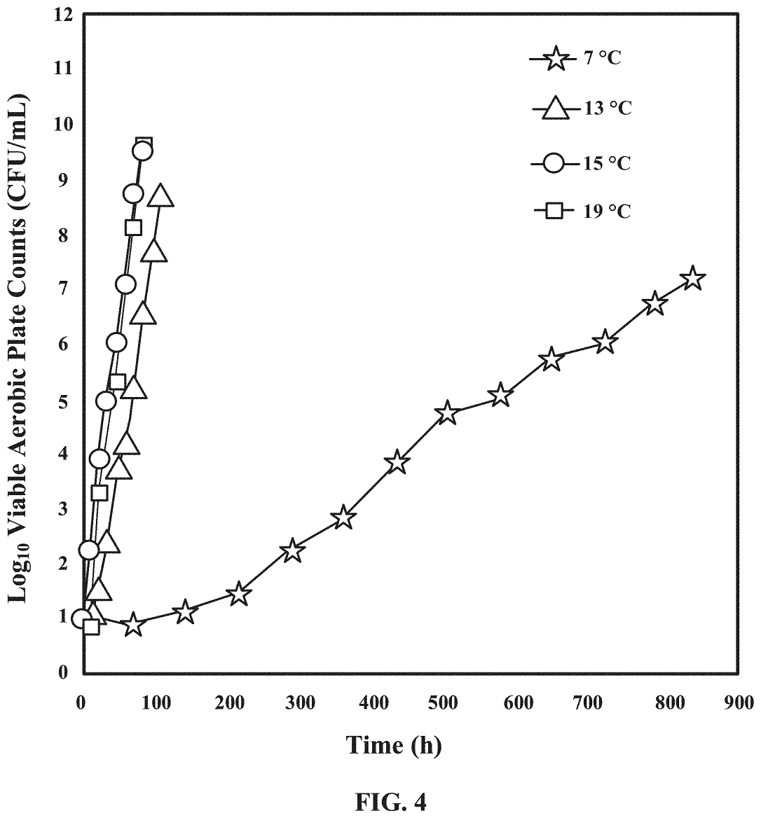

FIG. 4 shows log₁₀ aerobic plate counts (APCs) for milk stored at 19, 15, 13, and 7±1° C. On day 0 (control) the APCs were below 1.0 log₁₀ CFU/mL at each storage temperature, but samples stored at higher temperatures 19, 15 and 13° C. had greater APC log₁₀ counts than those stored at 7° C. Bacteria grew more rapidly at 19, 15, and 13° C. and had APCs of 7.0 log₁₀ CFU/mL after 24, 48, and 72 h, respectively. These measures were chosen because a viable bacterial count is one of the most commonly used tests to monitor and evaluate the shelf life and quality of milk in addition to many other food materials and beverages. Most studies report that the end of shelf life is reached when microbial populations in milk reach 5.0 to 7.0 log₁₀ CFU/mL; however, lower levels may cause sensory changes that can be detected by consumers as spoilage.

The primary factor in milk spoilage is bacterial activity resulting in a loss of sensory quality. In the tested milk, spoilage began within hours of storage as illustrated in FIG. 4. In milk held at 19, 15 and 13° C., the APC surpassed US Food and Drug Administration (FDA) limits within 48, 60 and 72 h, respectively consistent with other studies. The APCs of pasteurized milk at 7° C. showed slower bacterial growth, at 3.1 log₁₀ CFU/mL until 18 days similar to the 21 days observed by others.

VOCs were detected and identified in whole milk using headspace GC-FID analysis at different storage temperatures as shown in Table 1 as follows:

TABLE 1
Mean ± SD for volatile compounds (ng/mL) in pasteurized milk
samples over the shelf life at four storage temperatures.

| 19° c. | | Storage time (Days) | | | | | |
|---|---|---|---|---|---|---|---|
| Compound | | 1 | | 2 | | 3 | |
| Acetaldehyde | UD ± N/A | 2113 ± 125 | | 22560 ± 654 | | 25503 ± 1705 | |
| Acetone | 3210 ± 568 | 7627 ± 1012 | | 6648 ± 2230 | | 3976 ± 4405 | |
| 1-Pentanol | UD ± N/A | UD ± N/A | | UD ± N/A | | UD ± N/A | |
| Butanone | 28.5 ± 4.5 | 1155 ± 118 | | 3253 ± 294 | | 4231 ± 690 | |
| Ethanol | UD ± N/A | 1075 ± 54.1 | | 50 ± 39.8 | | 449 ± 86 | |
| Hexanal | 24.2 ± 13.8 | 1105.6 ± 112 | | 3652 ± 165 | | 3052 ± 515 | |
| 150 C. | | Storage time (Days) | | | | | |
| Compound | | 1 | | 2 | 3 | | 4 |
| Acetaldehyde | UD ± N/A | 425 ± 11 | | 15326 ± 452 | 22154 ± 632 | | 30232 ± 1020 |
| Acetone | 3034 ± 678 | 4462 ± 47.4 | | 4867 | 3527 ± 1497 | | 2842 ± 1034 |
| Butanone | 28.5 ± 6.3 | 5625 ± 68.0 | | 2077 ± 107 | 1525 ± 16.6 | | 1123 ± 105 |
| Ethanol | UD ± N/A | 8.86 ± 0.12 | | 43.0 ± 0.81 | 10.7 ± 2.2 | | 487 ± 19.9 |
| Hexanal | UD ± N/A | 927 ± 89.5 | | 2916 | 202 | | 1524 ± 274 |
| 130 C. | | Storage time (Days) | | | | | |
| Compound | | 1 | 2 | 3 | 4 | 5 | 6 |
| Acetaldehyde | UD ± N/A | UD ± N/A | 1625 ± 122 | 10203 ± 231 | 18065 ± 313 | 23653 ± 452 | 30215 ± 1090 |
| Acetone | 3034 ± 677 | 3480 ± 359 | 3918 188 | 4961 ± 138 | 9121 ± 54 | 8560 ± 227 | 6947 ± 231 |
| 1-Pentanol | UD ± N/A | UD ± N/A | UD ± N/A | 122 ± 1.2 | 653 ± 16.3 | 1325 ± 25.1 | 2012 35.2 |
| Butanone | UD ± N/A | 78.2 ± 5.2 | 1026 ± 235 | 1255 ± 225.3 | 2060 ± 254 | 19548 ± 432 | 1590 ± 222 |
| Ethanol | UD ± N/A | UD ± N/A | 42.5 ± 49.9 | 49.9 ± 4.2 | 22.3 ± 0.3 | 18.8 ± 5.1 | 654 ± 108 |
| Hexanal | UD ± N/A | UD ± N/A | UD ± N/A | 2326 ± 225 | 2965 ± 325 | 2589 ± 415 | 2059 ± 62 |
| TC | | Storage time (Weeks) | | | | | |
| Compound | | 1 | 2 | 3 | 4 | 5 | 6 |
| Acetaldehyde | UD ± N/A | UD ± N/A | UD ± N/A | UD ± N/A | UD ± N/A | UD ± N/A | UD ± N/A |
| Acetone | 3210 ± 568 | 3324.4 ± 166 | 3340 ± 1151 | 3420 ± 53 | 3103 ± 338 | 3014 ± 242 | 3186 ± 225 |
| Butanone | 28.4 ± 4.5 | 46.0 ± 34.6 | 47.1 ± 34.6 | 45.2 ± 3.1 | 14.8 ± 2.5 | UD ± N/A | UD ± N/A |
| Ethanol | UD ± N/A | UD ± N/A | 19.9 ± 4.5 | 5.1 ± 7.3 | UD ± N/A | UD ± N/A | UD ± N/A |
| Hexanal | UD ± N/A | 60.6 ± 10.8 | 33.9 ± 16.4 | 29.9 ± 0.5 | 24 ± 0.2 | 27.2 ± 0.3 | 38.1 ± 8.9 |
| 1-Pentanol | 3.6 ± 0.7 | 3.3 ± 0.2 | 3.3 ± 1.6 | 4.0 ± 0.1 | 2.6 ± 0.2 | 469 ± 3.8 | 686 ± 40.7 |

On day 0, 250 ml of whole milk was collected as a control. Low concentrations of butanone, acetone, and I-pentanol were present in the control at day 0. Similar compounds are found at all storage temperatures with ketones and aldehydes at the highest concentrations. Ketones were the most abundant compounds found in this study with acetone being the main constituent. In addition to the compounds identified at time 0, several others (e.g., acetaldehyde, butanone, hexanal, 1-pentanol, ethanol, acetic acid, butyric acid, and hexanoic acid) were identified at the highest storage temperature (19° C.). A similar volatile profile was found in milk stored at 15° C. The VOCs at this storage temperature resulted in low amounts at day 0, while the VOC profile increased with time of storage with the production of butanone, ethanol, hexanal, and 1-pentanol, as well as acetic acid, butyric, and hexanoic acid (see TABLE 1) similar to previous findings.

The VOC profile for milk stored at 13° C. is similar to milk stored at 15 and 19° C. As shown in TABLE 1 above, acetone and butanone concentrations remain constant throughout storage at 13° C., while ethanol is higher on day 2 of storage, decreasing sharply over the rest of the storage period. Similar volatile profiles were detected in the headspace of samples at 7° C. with milk remaining in good condition over an approximately 3-week storage period.

TABLE 1 also summarizes the key VOCs detected at 7° C. Several VOCs form after 5 weeks of storage, including butanone, ethanol, hexanal, 1-butanol, ethyl octanoate, and 2-methyl butyrate. In addition, acetic, butyric, hexanoic, and octanoic acids were detected after 30 days. Bacterial growth in milk at each of the storage temperatures is associated with compounds such as acetone, acetaldehyde, I-hexanol, butanal, hexanal ethanol, acetic acid, butyric, and hexanoic acid and is similar to earlier findings. This confirms a relationship between bacterial growth and VOCs, with VOC concentrations remaining relatively unchanged until microbial numbers reach 5.0 to 7.0 log₁₀ CFU/mL (end of shelf life). Other studies have reported VOC formation at 7.0-8.0 log₁₀ CFU/mL in foods.

### Colorimetric Sensor Response

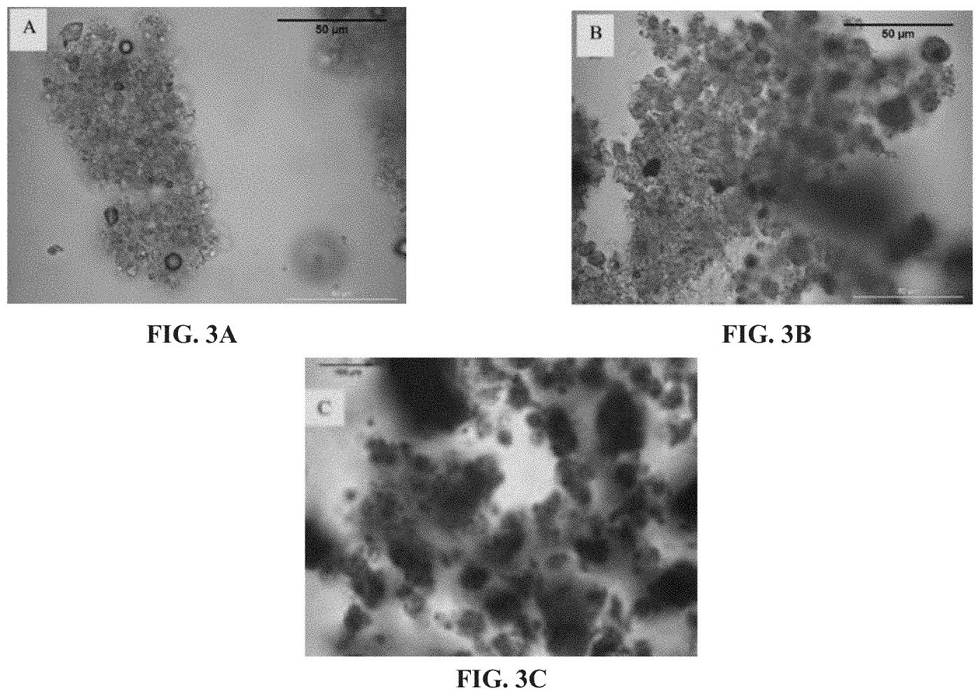

VOCs generated by microbes in the milk interact with the nanosensor to produce a color change from pink to purple. FIGS. 3A-3C show magnified images of SiO₂ nanoparticles, wherein FIG. 3A in particular shows uncoated nanoparticles, FIG. 3B shows Schiff's reagent coated nanoparticles, and FIG. 3C, while not expressly shown in the non-color figure, resulted in a purplish hue indicating that the nanoparticles reacted with spoiled milk VOCs.

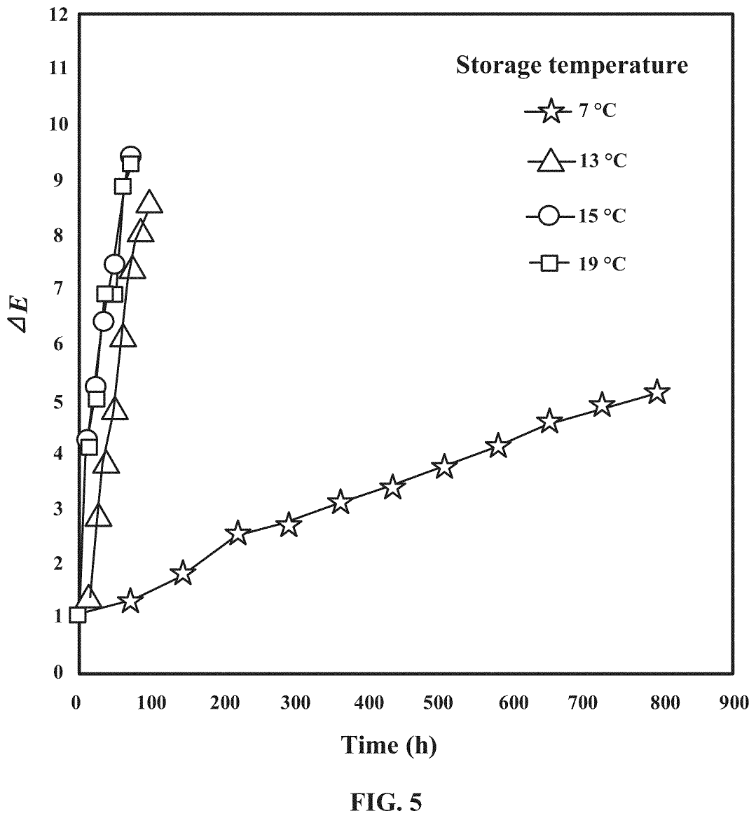

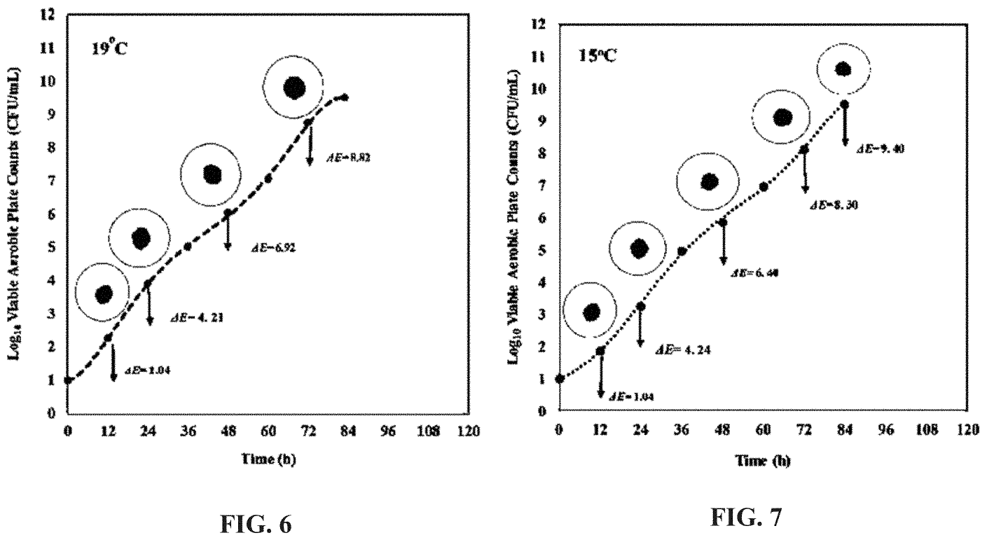

FIG. 5 illustrates color changes of Schiff's reagent coated nanoparticles over time for milk at each of four storage temperatures, wherein the ΔE scale is used to quantify differences between colors and wherein a ΔE value of 6 indicated spoilage. During the first 12 h of milk storage, no significant color change was detected (ΔE was 3.4 at 19° C.). After that, the nanosensor steadily changed from pink to light purple until 24 h, when ΔE was greater than 6 a generally recognized point at which a visual color change is distinctive and easily detected by the human eye. However, to the eye, the sensor provided a more intense color change and provided a good visual indication of spoilage at the end of storage when ΔE was greater than 9, as generally indicated in FIG. 6. The onset of color change was detected at 48 h for milk stored at 15° C. when ΔE reached 5.83. A color change was clearly visible at the end of shelf life (72 h) (FIG. 7). VOCs can react with Schiff's reagent and lead to a color change to magenta or purple. The Schiff's reaction involves the addition of a nucleophile group of the Schiff's reagent to the carbonyl group of an aldehyde. Because this nucleophile is extremely bulky, a ketone, which is more sterically crowded than an aldehyde at the carbonyl carbon, reacts with Schiff's reagent to a lesser extent. Schiff's reagent itself has a limited system of conjugation, the adduct with aldehydes have an extended system of conjugation, resulting in a highly colored compound. Other sterically restricted compounds will react in a similar manner and can be incorporated in the detection of food spoilage-related VOCs.

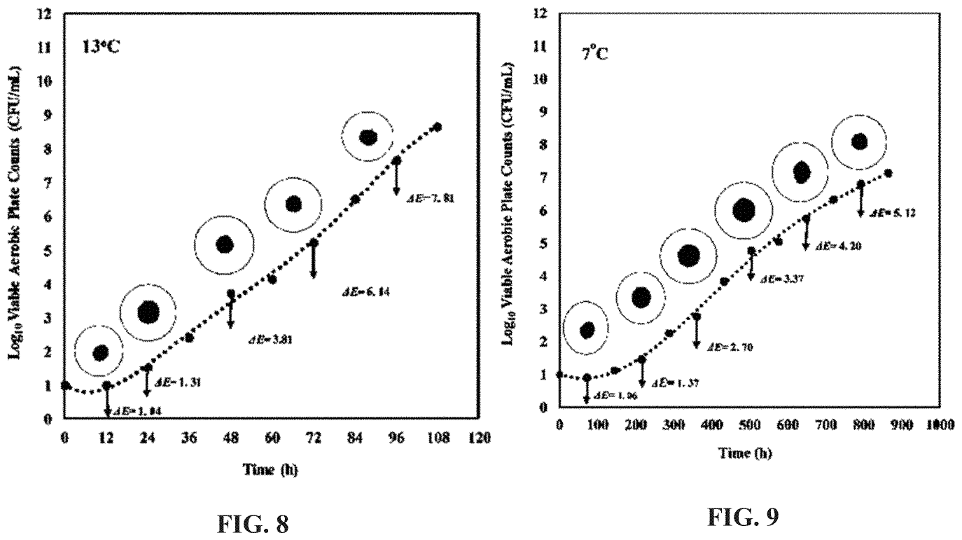

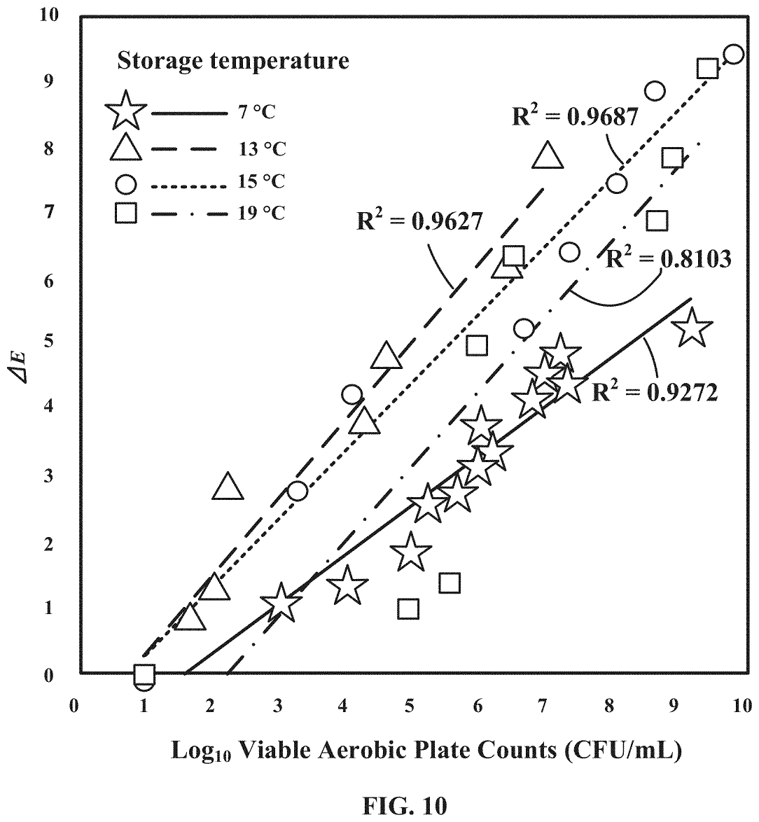

FIG. 8 shows aerobic counts and color change of sensor over milk shelf life at 13° C., wherein after 84 h of storage, the ΔE was 6.38, and a distinct visual color change was observed (FIG. 8). At the end of shelf life at 13° C., the ΔE was 8.04, with a pronounced color change. An interesting result was the relative lack of significant color change in the sample stored at 7° C.; the ΔE was stable at 3.6 until day 24, when it increased slightly (FIG. 9). The ΔE for this milk did not reach 6 by the end of the shelf life period. Thus, even though the ABC was 6.0 log, the color difference was imperceptible. This shows that the milk samples released VOCs at a relatively slow rate during low-temperature storage (TABLE 1). An increase in ΔE coincided with higher bacterial counts, and the color changes became more visible. In addition, the nanosensor response correlated well with microbial growth in milk samples stored at 19, 15, 13 and 7° C., with R² of 0.83, 0.94, 095 and 0.96, respectively (FIG. 10). Slopes describing the relationship between ΔE and higher bacterial counts were linear, and generally increased with higher storage temperature. Statistical differences in the slope were detected between 7° C. and both 13° C. (p=0.026) and 19° C. (p=0.0006) and between 15° C. and 19° C. (p=0.05).

Because of the concern with food waste along the distribution channel for refrigerated foods, colorimetric sensors incorporated in or on packaging will allow consumers to get a better idea of remaining shelf life at point of purchase and also while storing milk or other foods at home. This will enhance utilization and reduce food loss in markets where a significant amount of food and beverages are discarded because of problems with the cold chain. Because other food products that contain lipids and fermentable sugars have similar spoilage characteristics, this tested sensor is not limited to the application of fluid milk. With other reagents, it applies as well to food products in which VOCs are produced by spoiling processes, as is known to occur based in part on macronutrient composition. For example, heat-resistant and acid-tolerant Alicyclobacillus acidoterrestris produces aromatic volatile compounds such as bromophenol and guaiacol along with spoilage in several fruit juices, some of which are sold in fresh and pasteurized forms with shelf-life similar to milk.

### Mechanical Embodiments

Colorimetric sensors can be attached to food containers such that a reactive material is separated from the headspace by a gas-permeable membrane such as thin polyethylene or polypropylene. The material is selected to block liquid and solid contact while permitting communication of VOCs targeted for detection.

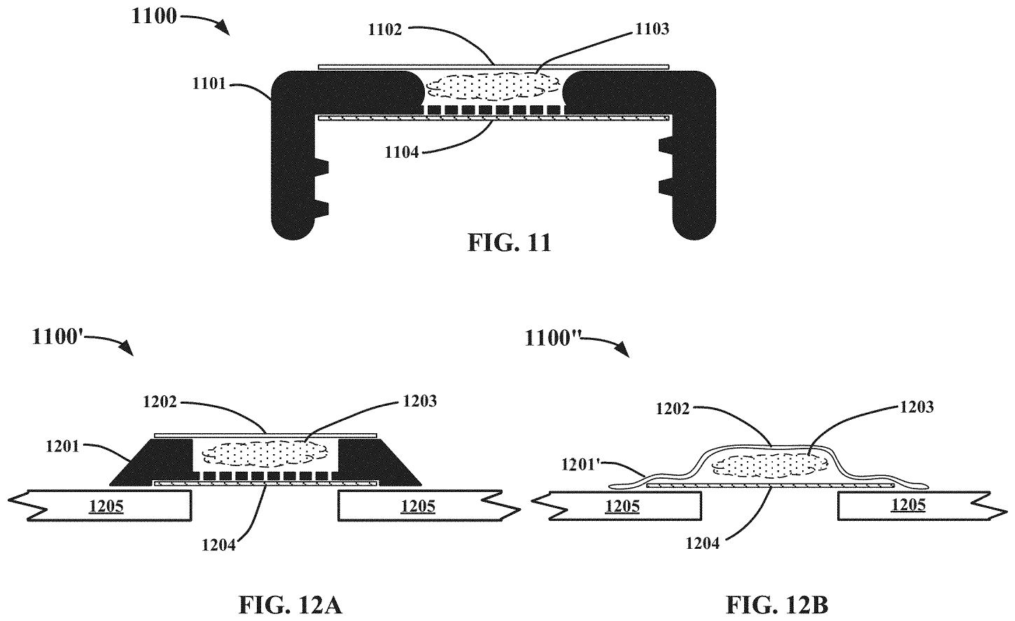

FIG. 11 illustrates a cross-section of an example sensor mount, as generally referenced by the numeral 1100, to provide sensing devices disclosed herein. In particular, the mount 1100 includes a threaded screw cap 1101 with colorimetric sensor incorporated. A reagent coated substrate (not detailed) forms the sensor core material 1103 which is held in place by a viewing window 1102 bonded to the exterior of the cap, and a gas-permeable membrane 1104 bound to the interior of the cap. The design of the threaded screw cap 1101 (vessel) enables the reagent coated substrate to be positioned adjacent to the food content/headspace in a food package. The sensor core may rest in a through hole or partial well within the cap. If in a partial well, gas-passages can be formed or cut in the cap to allow for headspace gases (or food content gases if no headspace) to reach the sensor core via the gas-permeable membrane. Such a design can help readability of colorimetric sensor by providing a cap material (e.g., plastic) of consistent color as a background for the sensor material.

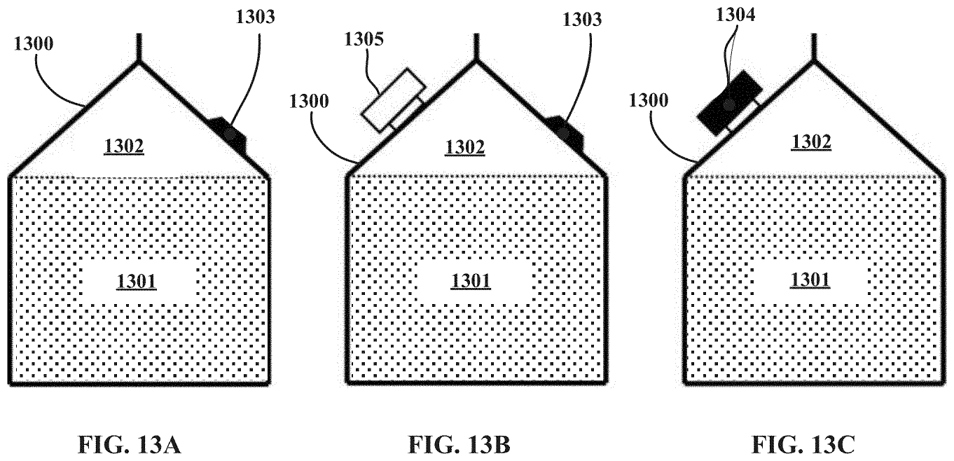

FIGS. 12A-B show additional cross-sections of example sensor mounts (vessels), referenced respectively as 1100′ and 1100″, which can be fixedly or removably coupled to a food package separate from its closure system, wherein FIG. 12A shows a rigid form, and FIG. 12B shows a flexible mount. In both variants, a clear non-permeable film 1202 provides a viewing window of the sensor core material 1203 while also operating as an oxygen barrier and a gas-permeable membrane 1204 separates the sensor core material 1203 from the headspace (e.g., see reference 1202 in FIGS. 13A-C) within the package (e.g., food package) 1205. As both variants are mounted over an aperture which is punched or otherwise formed in the food package as understood by those of ordinary skill in the art. A rigid mount 1201, as shown in FIG. 12A, can be configured with a containment region (not denoted) (e.g., a well or hole) to hold the sensor core. The rigid mount 1201 can be any inert material, such as, but not limited to, plastic, and offers often, but not necessarily, flat surfaces for hermetically bonding to layers 1202, 1204, and the package 1205 by adhesive, plastic welds, or other methods as known in the art. While a rigid mount 1201 is beneficial, a flexible mount (denoted as 1201′) can also be utilized in place of a rigid platform 1201, with layers (e.g., plastic) bonded directly to each other. Alternatively, the window film layer can be selected by a material known in the art which bonds well to the gas-permeable membrane, is sufficiently thick to provide security and durability, and is flexible enough to form a pocket for the sensor core material. As disclosed throughout but to be reiterated, the sensor mounts (vessels) enables the reagent coated substrate to be positioned adjacent to the food content/headspace in a food package.

Beyond use on factory packaged foods, sensor forms illustrated in FIG. 11 and FIGS. 12A-B can also be applied in contexts where a consumer acquires sensors separately from prepared food products. For example, threaded cap-based sensors can be distributed separately in sealed packaging or a peel-off form and added to consumer opened packages. Other forms can be fitted to pitchers or other reusable food storage containers. Similarly, flush mount sensors can be used in the context of baby bottle kits, where caregivers desire to store breast milk and monitor its freshness. In such an example embodiment, disposable sensors may be fitted with self-adhesive (peel-and-stick) or other means to temporarily attach to modified bottles or other food containers. Sensors can alternately be coupled using a pressure-fit snap or slide, which can include a compression seal, lips, and/or detents at one or both ends of the slide mechanism to lock sensors in place.

FIGS. 13A-C illustrate variations using a cross-section of a typical milk or juice carton, 1300, with a headspace 1302 above the product (food) 1301. FIG. 13A in particular, shows a carton without a cap closure system, where the colorimetric sensing device 1303 is simply mounted or built-in as a stand-alone unit (vessel) as shown in FIGS. 13A and 13B. Mounting may be by adhesive (e.g., removably coupled), welding as known in the art, or other means suitable to maintain package integrity. FIG. 13B shows that even where there is a cap 1305 for a typical typical milk or juice carton 1300, the sensing device 1303 can be independently positioned. This design can account for situations where a separate non-permeable tamper seal is required under the cap 1305. FIG. 13C shows the sensing device 1304 built into a cap either as illustrated in FIG. 11 or in a suitable alternate configuration.

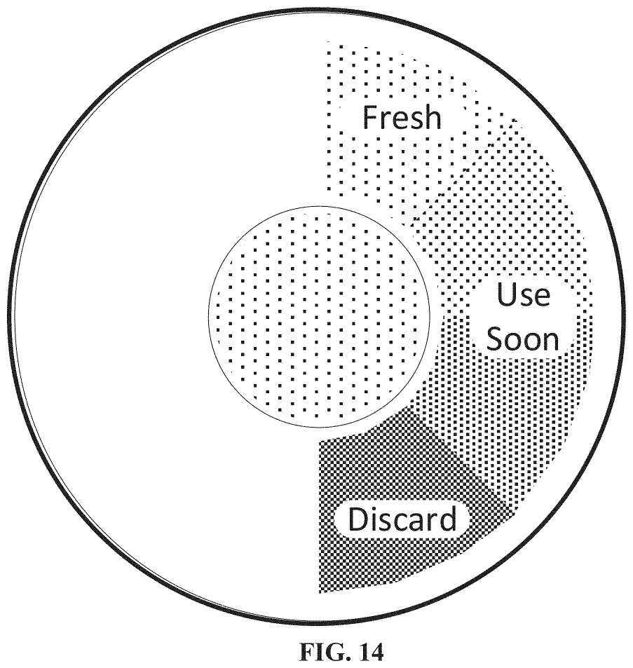

FIG. 14 provides an example package label which can be placed or printed adjacent to or around the colorimetric sensor. The sensor can be compared to a spectrum of colors by eye or a machine vision system (e.g., a camera coupled to a processor), with the printed colors allowing a degree of calibration across lighting conditions. In particular, wherein the spectrum of colors provides a visual indication of the color changes of the one or more reagents that measures the state of food spoilage deterioration. Further instructions can be printed on the label, including estimates of remaining freshness for various gradations under optimal storage conditions.

Sensors may be bonded similarly to other types of packaging with a headspace or where VOCs are anticipated to form and collect. Laboratory testing incorporated filter paper as a gas-permeable membrane, but other membranes (e.g., polyethylene, polypropylene films) which are configured to be gas-permeable while preventing liquid or solid passage can also be utilized when warranted. Such a membrane protects both the food product and the substrate from direct liquid or solid contact with each other. It is also understood that a membrane can include micro-perforations to achieve desired permeability.

Appropriate sensor reagents are selected based on the desired spoilage indicator (e.g., aldehyde concentration) and colorimetric characteristics, and then attached (e.g., functionalized) to a substrate. Substrates are selected based on surface-area-to-volume and other factors to include ease of reagent bonding, durability, and visibility. Some options include nanoparticles, natural and manufactured fibers, polymer sheets, and papers. Sensors include a portion of the coated substrate held in a vessel adjacent to the food content in a package, often adjacent to a headspace of a food package that captures the Volatile organic compounds (VOCs). This may be within or separate from the closure mechanism. The vessel may include a viewing window for reading color, or be comprised of a transparent or a translucent material, or allow alternate means of reading, such as a built-in color sensor and micro-transmitter.

While the invention has been described in terms of its example embodiments, those skilled in the art will recognize that the invention can be practiced with modification within the spirit and scope of the appended claims. Accordingly, the present invention should not be limited to the embodiments as described above, but should further include all modifications and equivalents thereof with the spirit and scope of the description provided herein.

We claim:

1. A sensing device for detecting food spoilage in a package, comprising:
a substrate;
one or more reagents coupled to one or more surfaces of the substrate;
a vessel configured with a viewing window and a gas-permeable membrane, wherein the vessel is arranged to position the substrate adjacent to a food content within the package, and wherein the gas-permeable membrane is therebetween the substrate and the food content within the package; and
wherein the one or more reagents provides a visual indication of spoilage of the food content.

2. The sensing device of claim 1, wherein the substrate is one or more of the group comprising: a nanoparticle substrate, a paper substrate, a fibers substrate, a polymer substrate, and a polymer sheet substrate.

3. The sensing device of claim 2, wherein the nanoparticle substrate is a plurality of silicon dioxide nanoparticles.

4. The sensing device of claim 1, wherein the package includes a headspace therebetween the food content within the package and the gas-permeable membrane to capture volatile organic compounds (VOCs).

5. The sensing device of claim 4, wherein the captured VOCs includes at least one of: acetone, acetaldehyde, I-hexanol, butanal, hexanal ethanol, acetic acid, butyric, and hexanoic acid.

6. The sensing device of claim 1, wherein the one or more reagents comprise a Schiff's reagent.

7. The sensing device of claim 1, wherein the visual indication is a gradual color change of the one or more reagents that measures the state of food spoilage deterioration.

8. The sensing device of claim 1, wherein the one or more reagents coupled to the substrate are disposed within a containment region of the vessel, and wherein the one or more reagents coupled to the substrate are further interposed between the viewing window and the gas-permeable membrane.

9. The sensing device of claim 1, wherein the gas-permeable membrane comprises a filter paper membrane, a polyethylene membrane or a polypropylene membrane.

10. The sensing device of claim 1, wherein the vessel positioning the substrate is configured as a releasable and reclosable closure device for the package.

11. The sensing device of claim 10, wherein releasable and reclosable closure device for the package is configured as a cap.

12. The sensing device of claim 1, wherein the vessel is configured as a flexible mount or a rigid mount.

13. A disposable sensing device for detecting food spoilage, comprising:
a substrate;
one or more reagents coupled to one or more surfaces of the substrate;
a vessel configured with a viewing window and a gas-permeable membrane, wherein the vessel is arranged to position the substrate adjacent to a food content within a container, and wherein the gas-permeable membrane is therebetween the substrate and the food content within the container; and
a means of removably coupling the vessel to the container.

14. The disposable sensing device of claim 13, wherein the means of removably coupling the vessel to the container comprises one or more of: a self-adhesive coupling, a pressure-fit snap, and a pressure-fit slide.

15. The disposable sensing device of claim 14, wherein the means of removably coupling the vessel includes a compression seal.

16. The disposable sensing device of claim 13, wherein the substrate comprises at least one of: a nanoparticle substrate, a paper substrate, a fibers substrate, a polymer substrate, and a polymer sheet substrate.

17. The disposable sensing device of claim 13, wherein the nanoparticle substrate is a plurality of silicon dioxide nanoparticles.

18. The disposable sensing device of claim 13, wherein the container includes a headspace therebetween the food content and the gas-permeable membrane to capture volatile organic compounds (VOCs).

19. The disposable sensing device of claim 18, wherein the captured VOCs includes at least one of: acetone, acetaldehyde, I-hexanol, butanal, hexanal ethanol, acetic acid, butyric, and hexanoic acid.

20. The disposable sensing device of claim 13, wherein the one or more reagents comprise a Schiff's reagent.

21. The disposable sensing device of claim 13, wherein the vessel positioning the substrate is configured as a releasable and reclosable closure device for the container.

22. The disposable sensing device of claim 13, wherein the vessel is configured as a flexible mount or a rigid mount.
